(19) United States
(12) Patent Application Publication
(10) Pub. No.: US 2013/0197438 A1
(43) Pub. Date: Aug. 1, 2013

(54) Microfluidic Delivery Systems

(71) Applicants: Hongyi Yang, Nottingham (GB); Xiaogin Ma, Nottingham (GB)

(72) Inventors: Hongyi Yang, Nottingham (GB); Xiaogin Ma, Nottingham (GB)

(21) Appl. No.: 13/580,536

(22) PCT Filed: Feb. 21, 2011
(86) PCT No.: PCT/GB2011/000230

§ 371 Date: Feb. 4, 2013

(30) Foreign Application Priority Data
Feb. 22, 2010 (GB) 1002962.7

(51) Int. Cl. A61M 5/44

(52) U.S. Cl. CPC A61M 5/44
USPC 604/114; 604/113

(57) ABSTRACT

A microfluidic delivery system (10) includes a reservoir (3) to hold a predetermined volume of fluid, such as a dose of a drug. An outlet (5) allows delivery to a patient. A piston (15) can move in the reservoir (3) to dispense fluid through the outlet (5). Movement of the piston (15) is provided by an actuator (9) including a coil (19) of shape memory allow. Other examples use SMA wires. The SMA element (19) is controlled by an electrical current.

The present invention relates to microfluidic delivery systems, and particularly to automated microfluidic delivery systems, for delivering fluids. More particularly, but not by way of limitation, the invention relates to microfluidic drug delivery systems.

As used herein the term “microfluidic system” refers to a system arranged to deliver small amounts of fluid, for example, but not by way of limitation, less than 1 ml of fluid.

Microfluidic systems have wide applications in medicine, biochemical research, and food and environmental analysis, to name but a few examples. Proper handling and transmission of small amounts of liquid samples are critical for these applications. One major challenge in these applications is the development of a micro-actuation system that can reliably deliver automated operation, has low power consumption, is small in size and can be cost effectively mass produced.

For an example, for patients suffering from a long-term medical condition, such as diabetes, continual management of the patient's condition is necessary. Conventionally diabetics control their condition by self administering doses of insulin using a pen type injector. Such self-injection is usually unpleasant for the patient. Furthermore, it is generally not possible for a patient to administer doses as frequently as might be desired. There is evidence to suggest that conditions such as diabetes are best treated by continual monitoring of blood glucose levels, with corresponding continual administering of insulin doses, if required.

According to a first aspect of the invention there is provided a microfluidic delivery system comprising a reservoir operable in use to hold a predetermined volume of fluid, the reservoir comprising an outlet, the system further comprising a dispense means operable to move from a first position to a second position, that movement arranged in use to cause the fluid to exit the reservoir through the outlet, and a shape memory alloy actuator operatively connected to the dispense means and arranged so that a change in temperature of the actuator causes the dispense means to move from the first position to the second position, the actuator being further arranged so that the dispense means does not return to the first position.

The change in temperature may be from a first temperature below a predetermined temperature to a second temperature above the predetermined temperature. The actuator may be arranged so that the dispense means does not return to the first position when the temperature of the actuator falls below the predetermined temperature. The dispense means may stay in the second position when the temperature of the actuator falls below the predetermined temperature.

The change in temperature may be caused by an electrical current applied to the actuator. The actuator may be arranged so that the dispense means does not return to the first position when the electrical current is discontinued. The actuator may stay in the second position when the electrical current is discontinued.

The shape memory alloy actuator may be connected at a first end to the dispense means and a second end to a fixed point associated with, for example located on, the reservoir.

The shape memory alloy actuator may comprise one or more SMA coils. The shape memory alloy actuator may comprise one or more SMA wires.

The dispense means may comprise a valve. In use, the valve may substantially close the outlet in the first position and permit fluid to exit the reservoir through the outlet in the second position. The valve may comprise a plug.

The dispense means may comprise a piston. The piston may be operable in use to expel fluid from the outlet as the piston moves from the first position to the second position. The piston may be slidably held within the reservoir.

The outlet may comprise one or more micro-outlets, for example microslits, microchannels, micro pores or micro tubes. In use, fluid may be retained in the reservoir by means of surface tension.

The delivery system may comprise first and second dispense means having a shape memory alloy actuator operatively connected between them, the such that a change in temperature of the actuator causes each of the dispense means to move from a first position to a second position.

The delivery system may comprise first and second dispense means and first and second shape memory alloy actuators, each actuator being arranged to move a respective dispense means from a first position to a second position.

The first dispense means may comprise a valve, such as a plug. The second dispense means may comprise a piston.

The first and second dispense means may each comprise a respective piston. Each piston may be connected to a respective lever, each lever operable in use to rotate around a pivot so as to cause the piston to move from the first position to the second position. The pivot may be common to both levers.

The outlet may comprise a closure.

According to the second aspect of the invention there is provided a microfluidic delivery system comprising a reservoir operable in use to hold a predetermined volume of fluid, the reservoir comprising an outlet, the system further comprising a dispense means operable to move from a first position to a second position, the movement arranged in use to cause the fluid to exit the reservoir through the outlet, and a shape memory alloy actuator operatively connected to the dispense means and arranged so that a change in temperature of the actuator causes the dispense means to move from the first position to the second position, wherein the outlet comprises a micro-outlet, such that in use fluid is retained in the reservoir by means of surface tension when the dispense means is in the first position.

The micro-outlet may comprise one or more microslits, microchannels, micro pores or micro tubes.

According to a third aspect of the invention there is provided a microfluidic delivery system comprising a reservoir operable in use to hold a predetermined volume of fluid, the reservoir comprising an outlet, the microfluidic delivery system further comprising a valve operable to move from a first position in which the valve substantially closes the exit to a second position in which in use a fluid is able to exit the reservoir through the outlet, the system further comprising a shape memory alloy actuator operatively connected to the valve and arranged so that a change in temperature of the actuator causes the valve to move from the first position to the second position.

According to a fourth aspect of the invention there is provided a microfluidic delivery system comprising a reservoir operable in use to hold a predetermined volume of fluid, the reservoir comprising an outlet, the system further comprising a pair of dispense means each movable from a first position to a second position so as to cause the fluid to exit the reservoir through the outlet, wherein the system further comprises one or more shape memory alloy actuators operatively connected to one or both of the dispense means and arranged so that a change in temperature of the or each actuator causes each of the dispense means to move from the first position to the second position.

The pair of dispense means may comprise a pair of pistons. Each piston may be connected to a respective lever, each lever operable in use to rotate around a pivot so as to cause the piston to move from a respective first position to a respective second position.

It will be appreciated that features described with reference to the first aspect of the invention might also be implemented in one or more of the other aspects of the invention.

The present invention will now be described, by way of example only, with reference to the accompanying drawings, in which:

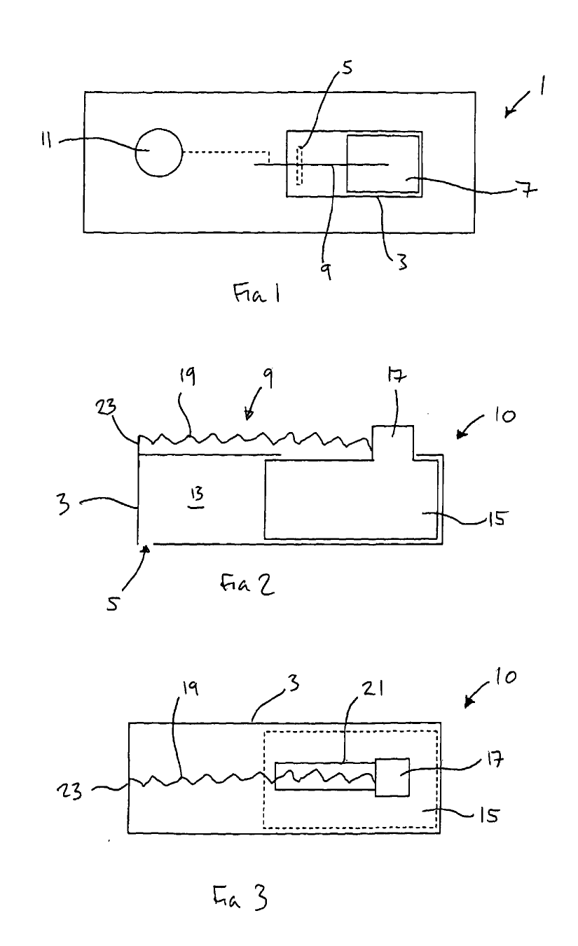

FIG. 1 shows a schematic view of a microfluidic delivery system;

FIG. 2 shows a cross-sectional view of an embodiment of a microfluidic delivery system in a first position;

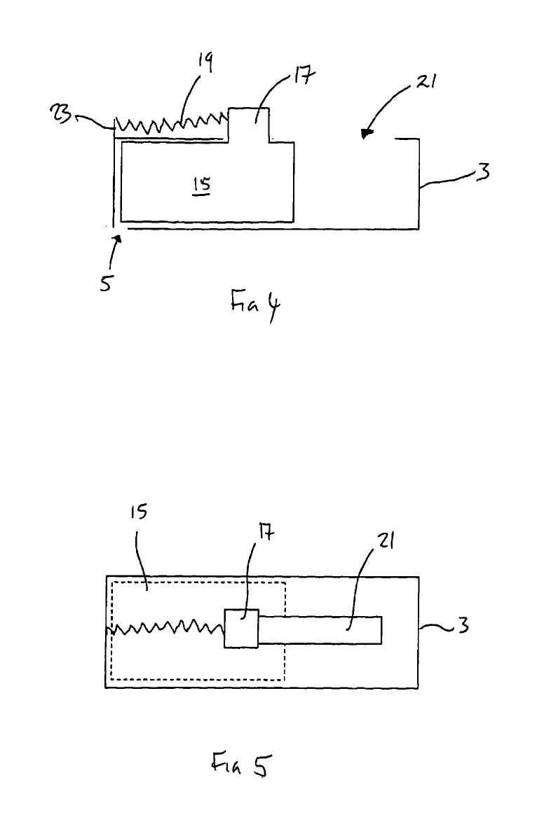

FIG. 3 shows a plan view of the microfluidic delivery system of FIG. 2 in the first position;

FIG. 4 shows a cross-sectional view of the microfluidic delivery system of FIG. 2 in a second position;

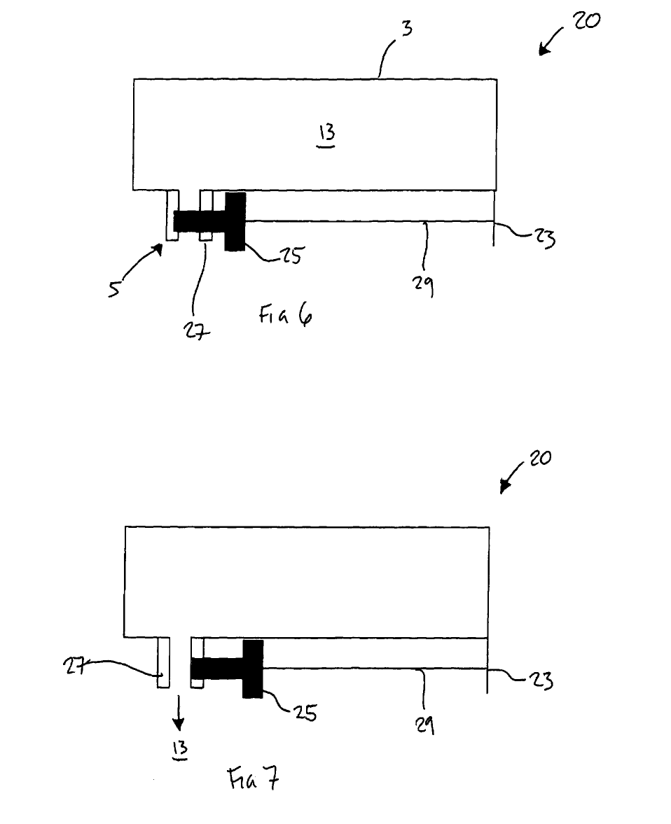

FIG. 5 shows a plan view of the microfluidic delivery system of FIG. 2 in the second position;

FIG. 6 shows a cross-sectional view of another embodiment of a microfluidic delivery system in a first position;

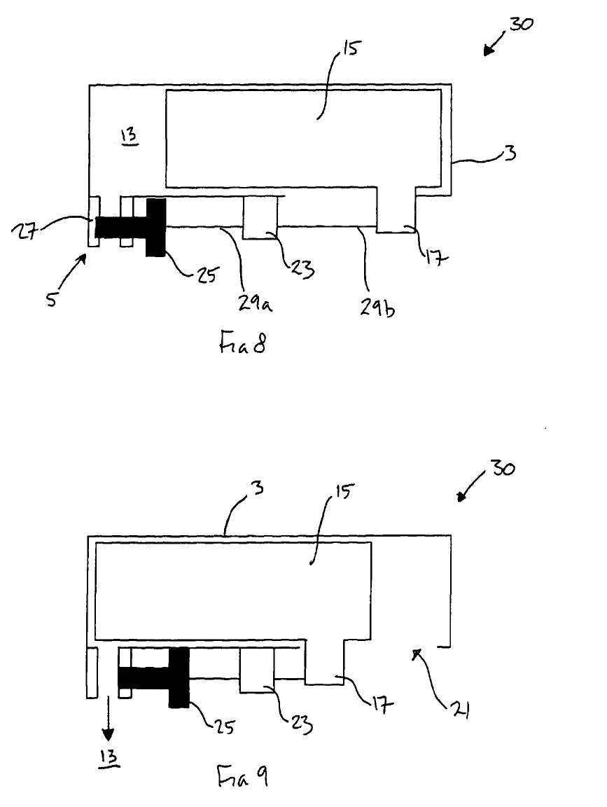

FIG. 7 shows a cross-sectional view of the microfluidic delivery system of FIG. 6 in a second position;

FIG. 8 shows a cross-sectional view of yet another embodiment of a microfluidic delivery system in a first position;

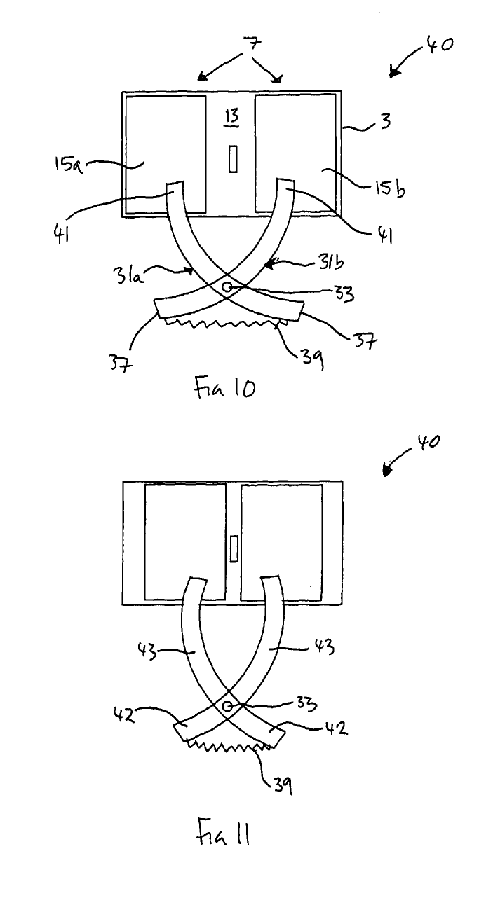

FIG. 9 shows a cross-sectional view of the microfluidic delivery system of FIG. 8 in a second position;

FIG. 10 shows a plan view of a further embodiment of a microfluidic delivery system in a first position;

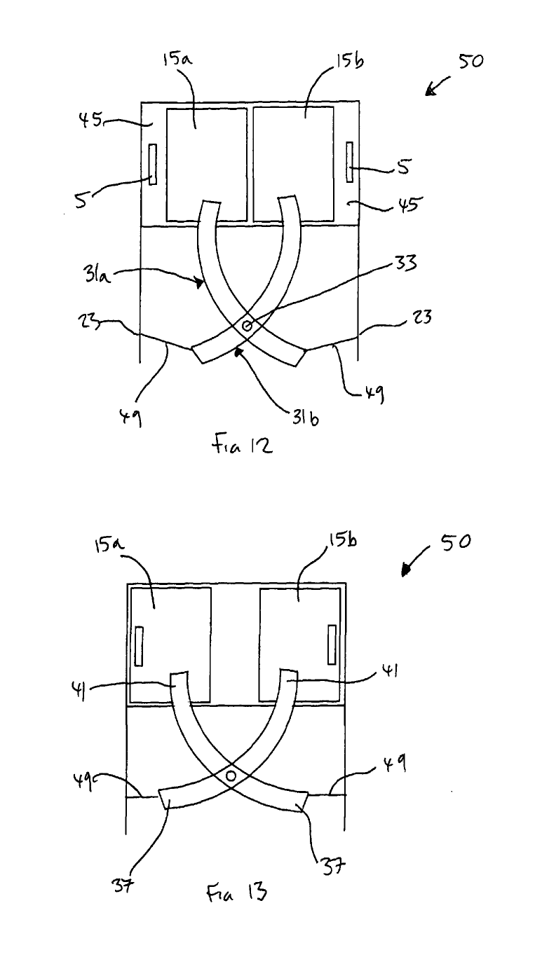

FIG. 11 shows a plan view of the microfluidic delivery system of Figure and in a second position;

FIG. 12 shows a plan view of another embodiment of a microfluidic delivery system in a first position; and

FIG. 13 shows a plan view of the microfluidic delivery system of FIG. 12 in a second position.

Referring to FIG. 1, a microfluidic delivery system 1 is shown. The microfluidic delivery system 1 includes a reservoir 3 which is operable in use to hold a predetermined volume of a fluid, for example a predetermined dose of a drug, or of a sample to be tested. The fluid could be liquid, cream or gel, for example. The reservoir comprises an outlet 5 through which fluid from the reservoir can be delivered to a patient. A dispense means 7, in this case in the form of a plunger or piston, is operable to move from a first position to a second position, that movement being arranged to cause the fluid to exit the reservoir 3 through the outlet 5.

The movement of the dispense means 7 is triggered by an actuator 9. The actuator comprises a shape memory alloy material, which may be in the form of a wire, or may be in the form of a coil, or may be arranged in any other suitable shape.

It is a property of shape memory alloy materials that when the temperature of such a material is raised above a predetermined temperature, the material undergoes a structural change. That structural change can also result in a dimensional change, because, when a shape memory alloy material is cooled to below its transition temperature, it can be easily deformed, and so moulded into other shapes. When the material is heated to above the transition temperature, the change in material structure causes the material to return to its original shape.

In the fluid delivery device shown in FIG. 1 the structural change of the SMA actuator is triggered by an electric current produced by a battery 11.

It will be noted that no closure or valve is shown as preventing fluid in the reservoir from exiting the outlet. That is because, in this example, the outlet comprises a micro-outlet in the form of a microslit. The smallest dimension of the microslit is chosen so that fluid in the reservoir cannot escape from the reservoir through the microslit at a normal atmospheric pressure. Instead, fluid is held within the reservoir by surface tension of the fluid is itself. However, fluid can be pushed from the reservoir through the slit by pressure from the dispense means. The exact dimensions of the microslit depend on the properties of the fluid.

Theoretically, the dimension of the microslit can be estimated by the Young-Laplace equation:

$$Δ p = γ ( \frac{1}{{R}_{X}} + \frac{1}{{R}_{Y}} )$$

However, various non-linear effects and measurement error mean that in practice this equation is invalid, such that it is better to determine the optimal microslit dimension experimentally.

The outlet of such a device might be provided with a protective layer or closure during storage, to prevent contamination and evaporation of the fluid, for example, which can be peeled away or removed when the device is to be used.

Referring now to FIGS. 2 to 5, an embodiment of a microfluidic device 10 is shown. The device comprises a reservoir 3 in which a predetermined volume of fluid 13 is held. The reservoir comprises an outlet 5 in the form of a microslit. A movable piston 15 acting as a dispense member is slideably held within the reservoir so that it can be moved from a first position shown in FIGS. 2 and 3 to a second position shown in FIGS. 4 and 5.

The piston 15 comprises a slider 17 which protrudes from the reservoir through a slot 21.

An SMA actuator 9 comprising an SMA coil 19 has a first end connected to a fixed point 23 provided on the reservoir, and a second end connected to the slider 17. The SMA coil 19 is also connected to a source of electrical current, such as a battery (not shown).

In use, when it is desired to deliver the fluid from the reservoir (for example at a predetermined time, or in response to, or to a measurement recorded by monitoring device a signal from a controller), an electric current is applied to the SMA coil 19, which causes the SMA coil to contract. The contraction of the SMA coil pulls the slider 17, and so the piston 15, towards the fixed point 23. The movement of the piston expels the fluid from the reservoir through the microslit. The walls of the reservoir act as a guide to control the sliding movement of the piston. In the example shown the reservoir and piston have a rectangular cross-section. However, it will be appreciated that any shape of reservoir and piston might be used as required, for example a reservoir having a circular cross-section.

If required, an SMA wire could be used instead of an SMA coil. However, because of its helical structure, an SMA coil can produce a much greater stroke than an SMA wire of similar length. For example, while an SMA wire might change in dimension (and so in length) by 8% when a current is applied, an SMA coil can be arranged to change in lateral dimension by 200%. Thus it is possible to move a piston through a greater distance using an SMA coil than using an SMA wire of the same starting length.

The electric current required for actuation may be in the range from 50 mA to 1 A, for example, and will depend on the dimensions of the SMA material used.

Referring now to FIGS. 6 and 7, an alternative microfluidic delivery system 20 is shown. As before, the microfluidic delivery system 20 includes a reservoir 3 comprising a predetermined volume of fluid 13. The reservoir comprises an outlet 5.

The outlet comprises a tube 27 which is sealed by a valve, such as a knife gate valve, which in this example is in the form of a plug 25. The plug 25 is movable from a first position in which the outlet is blocked or otherwise sealed (as shown in FIG. 6), to a second position in which the outlet is open so that fluid from the reservoir can exit the reservoir through the outlet (as shown in FIG. 7).

An actuator in the form of an SMA material, in this case a SMA wire 29, is arranged to move the plug 25 from the first position to the second position when an electric current is applied to the SMA wire. The SMA wire is connected at a first end to a fixed point 23 provided on the reservoir, and at a second end to the plug itself. The plug might be of any shape, the only requirement being that the plug should be equal to or slightly larger in size than the outlet, to better prevent the liquid inside the reservoir from leaking.

In use, when it is desired to deliver the predetermined volume of fluid that is held in the reservoir, an electric current is applied to the SMA wire 29, causing it to contract. The contraction of the SMA wire causes the plug to move from the first position in which the outlet is obstructed to the second position in which the flow of fluid from the reservoir is substantially unimpeded.

A microfluidic delivery system having this construction is simpler to manufacture than the device shown in FIGS. 2 to 5, as less parts are required. In addition, as larger volume of fluid can be held in the reservoir, because it is not necessary to also accommodate a piston within the reservoir.

Referring to FIGS. 8 and 9, another microfluidic delivery device 30 is shown. The device is similar to that of FIGS. 6 and 7 in that the outlet 5 of the reservoir 3 is blocked or sealed by a plug 25 to hold fluid 13 within the reservoir 3. However, the device 30 is similar to that of FIGS. 2 to 5 in that it comprises a delivery means in the form of a piston 15.

In this embodiment two actuators are provided, in the form of SMA wires 29a and 29b. Both SMA wires are connected at a first end to a fixed point 23 on the reservoir, in the form of a protrusion extending from the reservoir. A second end of the first SMA wire 29a is connected to the plug, and a second end of the second SMA wire 29b is connected to a slider 17 provided on the piston.

In use, a current is applied to both SMA wires 29a and 29b, causing both wires to contract. As the wires contract, the plug 25 is pulled towards the fixed point 23, so as to open the outlet. At the same time, the slider 17 is pulled towards the fixed point 23, so as to cause the piston to slide within the reservoir, that movement forcing fluid 13 from the outlet.

If desired, SMA coils may be used instead of the SMA wire 29a or 29b. For example, SMA coils may allow a greater stroke length to be produced.

A delivery device of this construction might be particularly useful when it is desired to deliver a fluid having very low surface tension or large volume, where it would not be practical to rely on surface tension alone to hold the fluid within the reservoir.

Referring to FIGS. 10 and 11, yet another embodiment of a microfluidic delivery device 40 is shown.

As before, the delivery device 40 comprises a reservoir 3 in which a predetermined volume or dose of a fluid 13 is held. The reservoir includes a micro-outlet 5, which in this case is in the form of a microslit, but might also be a micro-channel, one or more micro pores or micro-tube.

The device also includes a delivery means 7 operable to move from a first position to a second position in order to expel the fluid 13 from the reservoir. In this case, the delivery device in fact includes a pair of delivery members in the form of pistons 15a and 15b. A pair of levers 31a and 31b are each connected to a respective piston. Each lever is connected to a pivot, which in this case is a shared pivot 33.

In use, each lever is arranged to rotate about the pivot 33 so as to cause its respective piston to slide within the reservoir from a first position (as shown in FIG. 10) to a second position (as shown in FIG. 11). The movement of the pistons from the first position to the second position is arranged to cause the fluid 13 held in the reservoir to be expelled from the reservoir through the microslit.

An SMA actuator, in this case a SMA coil 39, is connected between the free ends 37 of the two levers. In use, a current is applied to the SMA coil 39, which causes the coil to contract. The contraction of the coil pulls the two free ends of the levers closer together. As the ends of the levers are pulled closer together the levers rotate about the pivot causing the opposite ends 41 of the levers, which are connected to the pistons 15a and 15b, to also move closer together.

It will be seen that a driving portion 42 of the levers (which lies between the pivot and the free ends 37) is shorter than a driven portion 43 of the levers (which lies between the pivot and the opposite ends 41). With this arrangement it is possible to cause the pistons to move through a greater distance than would be possible using an SMA wire alone, because the scissor of arrangement of the levers amplifies the stroke of the SMA coil 39.

The levers 31a and 31b are curved, in this example, but could be designed in any shape depending and the shape desired for the end product.

Referring now to FIGS. 12 and 13, those show a microfluidic delivery device 50 similar to that of FIGS. 10 and 11. The device of FIGS. 12 and 13 differs from that of FIGS. 10 and 11 in that the reservoir 3 comprises two fluid holding regions 45, each of which is provided with the respective outlet 5.

The arrangement of the levers and dispense members is the substantially same as for the delivery device 40, except that in the first position the levers are close together and are drawn apart to move into the second position. Two SMA actuators in the form of SMA wires 49 are connected at a first end to a respective fixed point 23, and at a second end to a respective free end 37 of each lever.

In use, an electric current is applied to each SMA wire 49, which causes that wire to contract. As the wires contract, the free ends 37 of the levers are pulled apart. That force on the free ends of the levers causes the levers to rotate about the pivot point 33, causing the opposite ends 41, and so the pistons 15a and 15b, to also move apart. As the pistons move into the fluid holding regions 45, fluid 13 in those regions is forced out of the reservoir through the outlets 5.

It will be appreciated that a different fluid could be held in each fluid holding region 45. As an alternative to the construction shown in Figures, two individual reservoirs might be provided, rather than a single reservoir having two separate fluid holding regions.

All of the devices shown herein are simple to construct (for example by means of an automated assembly process) and inexpensive, and so are suitable for use as a single-use device. In such a single-use device, the device need not be arranged so as to return the dispense means (or the plug) to the first position after the fluid has been dispensed. That is, no biasing means is required to move the dispense means (or the plug) back to its original position.

In a single use device, the reservoir can be pre-dosed with fluid through the outlet, and no inlet is required. Again, this results in a device having a simple structure. In addition, it means that a patient cannot alter the dose of a medicament that has been prescribed by a medical professional.

In an alternative example, an inlet is provided and can be sealed after pre-dosing the reservoir, resulting in a single use device.

If it is required to make a device capable of multiple actuations, the SMA actuator might comprise a two-way SMA wire or coil (that is, a wire or coil made from an SMA material which is arranged to cycle between two predetermined shapes). Alternatively, two complimentary SMA wires or coils may be provided, one operable move the dispense means or plug in a first direction, and the other operable to move the dispense means or plug in a second, opposite direction.

A microfluidic delivery device such as the ones described herein can be incorporated, for example, in a patch to be worn on the skin of a patient, so that a drug can be delivered to the patient intradermally. Alternatively, a delivery device such as the ones described might be employed to accurately dispense small volumes of fluid into a testing or sample chamber.

in the example of a patient having diabetes, one or more doses of insulin can be delivered using such a system, meaning the patient does not have to self inject. Furthermore, delivery of the insulin may be automatic. For example, the patient's glucose level might be monitored, and when the glucose level increases beyond a critical level the delivery system might automatically deliver the drug to the patient. Thus the system might also include a monitoring device arranged in use to monitor a characteristic, such as a blood sugar level, of the patient. A system controller might be operable to cause the SMA actuator to deliver the fluid on receipt of an indication from the monitoring device. The indication might be, for example, that the characteristic has fallen below, risen above or reached, a predetermined value.

Various modifications may be made without departing from the scope of the invention. For example, where an SMA wire is shown, it could be replaced with an SMA coil, and vice versa. Other shapes of SMA material could also be used. Two or more SMA components, such as wires or coils, could be used in parallel, as part of a single actuator. All that is required is that the contraction of the SMA material produces sufficient displacement and force to push the required amount of liquid out of the reservoir/move the plug away from the outlet.

With reference to FIGS. 10 to 13, other shapes or designs of lever might be used. It will be appreciated that one lever might be used.

It will be appreciated that the temperature change, and associated structural change, of the SMA actuator may be triggered in any suitable way. For example the temperature change might be due to the application of electrical current (as discussed above), or electromagnetic waves, chemical reactions and/or change in ambient temperature.

Whilst endeavouring in the foregoing specification to draw attention to those features of the invention believed to be of particular importance it should be understood that the applicant claims protection in respect of any patentable feature or combination of features hereinbefore referred to and/or shown in the drawings whether or not particular emphasis has been placed thereon.

13-32. (canceled)

33. A microfluidic delivery system comprising a reservoir operable in use to hold a predetermined volume of fluid, the reservoir comprising an outlet, the system further comprising a dispenser operable to move from a first position to a second position, that movement arranged in use to cause the fluid to exit the reservoir through the outlet, and a shape memory alloy actuator operatively connected to the dispenser and arranged so that a change in temperature of the actuator causes the dispenser to move from the first position to the second position, the actuator being further arranged so that the dispenser does not return to the first position.

34. A microfluidic delivery system according to claim 33, in which the change in temperature is from a first temperature below a predetermined temperature to a second temperature above the predetermined temperature.

35. A microfluidic delivery system according to claim 34, in which the actuator is arranged so that the dispenser does not return to the first position when the temperature of the actuator falls below the predetermined temperature.

36. A microfluidic delivery system according to claim 35, in which the dispenser stays in the second position when the temperature of the actuator falls below the predetermined temperature.

37. A microfluidic delivery system according to claim 33, in which the change in temperature is caused by an electrical current applied to the actuator.

38. A microfluidic delivery system according to claim 37, in which the actuator is arranged so that the dispenser does not return to the first position when the electrical current is discontinued.

39. A microfluidic delivery system according to claim 38, in which the actuator stays in the second position when the electrical current is discontinued.

40. A microfluidic delivery system according to claim 33, in which the shape memory alloy actuator is connected at a first end to the dispenser and a second end to a fixed point associated with the reservoir.

41. A microfluidic delivery system according to claim 33, in which the shape memory alloy actuator comprises one or more SMA coils or wires.

42. A microfluidic delivery system according to claim 33, in which the dispenser comprises a valve.

43. A microfluidic delivery system according to claim 42, in which, the valve substantially closes the outlet in the first position and permits fluid to exit the reservoir through the outlet in the second position.

44. A microfluidic delivery system according to claim 42, in which the valve comprises a plug.

45. A microfluidic delivery system according to claim 33, in which the dispenser comprises a piston slidably held within the reservoir.

46. A microfluidic delivery system according to claim 45, in which the piston is operable in use to expel fluid from the outlet as the piston moves from the first position to the second position.

47. A microfluidic delivery system according to claim 33, in which the outlet comprises one or more micro-outlets, microslits, microchannels, micro pores or micro tubes.

48. A microfluidic delivery system according to claim 33, in which in use, fluid is retained in the reservoir by means of surface tension.

49. A microfluidic delivery system according to claim 33, in which the delivery system comprises first and second dispenser having a shape memory alloy actuator operatively connected between them, such that the change in temperature of the actuator causes each of the dispenser to move from a first position to a second position.

50. A microfluidic delivery system according to claim 33, in which the delivery system comprises first and second dispensers and first and second shape memory alloy actuators, each actuator being arranged to move a respective dispenser from a first position to a second position.

51. A microfluidic delivery system comprising a reservoir operable in use to hold a predetermined volume of fluid, the reservoir comprising an outlet, the microfluidic delivery system further comprising a valve operable to move from a first position in which the valve substantially closes the exit to a second position in which in use a fluid is able to exit the reservoir through the outlet, the system further comprising a shape memory alloy actuator operatively connected to the valve and arranged so that a change in temperature of the actuator causes the valve to move from the first position to the second position.

52. A microfluidic delivery system comprising a reservoir operable in use to hold a predetermined volume of fluid, the reservoir comprising an outlet, the system further comprising a pair of dispensers each movable from a first position to a second position so as to cause the fluid to exit the reservoir through the outlet, wherein the system further comprises one or more shape memory alloy actuators operatively connected to one or both of the dispensers and arranged so that a change in temperature of the or each actuator causes each of the dispensers to move from the first position to the second position.
